(19) United States
(12) Patent Application Publication
(10) Pub. No.: US 2016/0129192 A1
(43) Pub. Date: May 12, 2016

(54) SYRINGE HAVING MULTIPLE LUMENS

(71) Applicant: Steven Rothenberg, Marlboro, NJ (US)

(72) Inventor: Steven Rothenberg, Marlboro, NJ (US)

(21) Appl. No.: 14/929,286

(22) Filed: Oct. 31, 2015

Related U.S. Application Data
(60) Provisional application No. 62/078,300, filed on Nov. 11, 2014.

(51) Int. Cl. A61M 5/31

(52) U.S. Cl. CPC A61M 5/3137; A61M 5/3135; A61M 5/3134; A61M 2005/3139

(57) ABSTRACT

A syringe can be used to receive fluid from a patient. The syringe can include a syringe body, a first lumen disposed within an internal space of the syringe body, a second lumen disposed within the internal space of the syringe body, and a plunger slideably received along an internal surface of the second lumen. The syringe can also include a stem elongate along a first direction, a handle coupled to the stem, and a needle coupled to the syringe body. The syringe can be configured such that the plunger is moved through the second lumen along a first direction and the fluid is received into the first lumen along a second direction that is opposite the first direction.

### CROSS-REFERENCE TO RELATED APPLICATIONS

This application claims the benefit of U.S. Provisional Patent Application No. 62/078,300; filed Nov. 11, 2014; and entitled SYRINGE HAVING MULTIPLE LUMENS. The entire contents of Patent Application No. 62/078,300 are incorporated by reference herein.

### TECHNICAL FIELD

The disclosure is applicable to syringes, and more specifically to syringes having multiple lumens.

### BACKGROUND

Syringes can be used in various applications to collect fluid from people and animals. Conventional syringes can include a hypodermic needle disposed at a distal end and a plunger that fits tightly within a lumen of the syringe. The plunger can be pulled and pushed along an inner surface of the lumen. The needle can be inserted into the person or animal and when the plunger is pulled in a direction away from the needle, this can create negative pressure to thereby draw fluid into the lumen. When the plunger is pushed in a direction towards the needle, this can create a positive pressure to thereby expel the fluid through the needle or an orifice at the distal end of the syringe.

A drawback of conventional syringes is that it can be awkward for a user to pull the plunger in a direction away from the needle, while also applying force in an opposite direction to thereby maintain the position of the syringe with respect to the person or animal. A system or method is desired that remedies this drawback.

### SUMMARY

The disclosure describes a syringe that includes a syringe body elongate along a first direction, wherein the syringe body defines a proximal portion, a distal portion that is opposite the proximal portion, a sidewall that extends between the proximal and distal portions, and an internal space, a first lumen disposed within the internal space of the syringe body, a second lumen disposed within the internal space of the syringe body, a plunger slideably received along an internal surface of the second lumen, wherein the plunger defines a proximal surface and a distal surface that is opposite the proximal surface, and wherein the plunger is configured to move along a first direction and a second direction that is opposite the first direction, a stem elongate along the first direction, wherein the stem has a proximal end and a distal end that is opposite the proximal end, and wherein the distal end of the stem is coupled to the proximal surface of the plunger, wherein the stem is configured to move along the first direction and the second direction, a handle coupled to the proximal end of the stem, wherein the handle is configured to move along the first direction and the second direction, wherein the handle and plunger are configured to move from a first position to a second position, wherein when the handle and plunger are in the first position, the syringe defines a first length, wherein when the handle and plunger are in the second position, the syringe defines a second length, and wherein the second length is greater than the first length, and a needle coupled to the distal portion of the syringe body, wherein the needle is configured to receive fluid through a tip of the needle, and wherein the fluid moves from the needle into the first lumen.

In some embodiments the second lumen can be disposed within an internal space of the first lumen. The syringe body can define a first flow path between the first lumen and the second lumen. When the plunger and handle move from the second position to the first position, air can move from the first lumen through the first flow path and to the second lumen to thereby pull the fluid from the needle into the first lumen. As well, the first flow path can be disposed at a proximal portion of the syringe body.

The sidewall of the syringe body can define a second flow path. The second flow path can provide air flow path between the second lumen and the outside air. When the plunger and handle move from the second position to the first position, air can move from the second lumen through the second flow path to thereby pull the fluid from the needle into the first lumen.

In some embodiments the syringe further includes a seal disposed within an internal space of the second lumen along a proximal portion of the second lumen. The seal can slideably and sealably receive the stem. The seal can be configured to provide a vacuum-tight sealing engagement.

The first lumen can include a proximal portion and a distal portion that is opposite the proximal portion. Likewise, the second lumen can include a proximal portion and a distal portion that is opposite the proximal portion. In some embodiments, the distal portion of the first lumen can be distal to the distal portion of the second lumen. A first axis that is parallel to the first direction can thereby pass through at least a portion of the second lumen and at least a portion of the first lumen.

The disclosure also describes methods for removing fluid from a patient. Many methods can include obtaining a syringe having a syringe body, a first lumen disposed within an internal space of the syringe body; a second lumen disposed within the internal space of the syringe body, a plunger slideably received along an internal surface of the second lumen, a stem elongate along a first direction, wherein the stem can be configured to move along the first direction and a second direction that is opposite the first direction. The syringe can also include a handle coupled to the stem, wherein the handle is configured to move along the first direction and the second direction. The syringe can also include a needle coupled to the syringe body, wherein the needle is configured to receive the fluid through a tip of the needle. Methods can include pushing the handle of the syringe to thereby move the plunger along the first direction towards the needle. In response to pushing the handle of the syringe to thereby move the plunger along the first direction towards the needle, methods can include causing the fluid to flow into the first lumen, wherein the fluid flows from the tip of the needle into the first lumen along the second direction.

Some methods can also include causing the first lumen to fill with fluid. The fluid can enter the first lumen from the distal portion of the first lumen, and wherein as the handle is pushed, the fluid can rise from the distal portion of the first lumen towards the proximal portion of the first lumen.

Furthermore, as the fluid enters the needle, a top surface of the fluid can be located distal to the plunger. Methods can also include pushing the handle of the syringe along the first direction until the plunger is located distal to the top surface of the fluid.

Some methods can include causing at least a portion of the fluid to surround at least a portion of the second lumen. As well, some methods can include pulling the handle of the syringe to thereby move the plunger along the second direction. In response to pulling the handle of the syringe to thereby move the plunger along the second direction, some methods can include causing the fluid to flow from the first lumen to the tip of the needle along the first direction.

The syringe can include a seal disposed within an internal space of the second lumen along a proximal portion of the second lumen. The seal can slideably and sealably receive the stem such that the seal provides a vacuum-tight sealing engagement with the stem. Furthermore, some methods can include pushing the handle of the syringe to create a vacuum to thereby draw fluid into the first lumen.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a front view of a syringe in a first position, according to an embodiment.

FIG. 2 illustrates a front view of the syringe from FIG. 1 in a second position, according to an embodiment.

FIG. 3 illustrates a top down view of the syringe from FIG. 1, according to an embodiment.

FIG. 4 illustrates a front view of a syringe in a first position, according to another embodiment.

FIG. 5 illustrates a front view of the syringe from FIG. 4 in a second position, according to another embodiment.

FIG. 6 illustrates a top down view of the syringe from FIG. 4, according to another embodiment.

FIG. 7a illustrates a front view of the syringe from FIG. 1, whereby a top surface of fluid is located distal to a plunger, according to an embodiment.

FIG. 7b illustrates a front view of the syringe from FIG. 1, whereby the top surface of the fluid is located proximal to the plunger, according to an embodiment.

FIG. 7c illustrates a top down view of the syringe from FIG. 1, whereby at least a portion of fluid surrounds a second lumen.

FIG. 8 illustrates a flow-chart of a method of using a syringe, according to various embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Device Embodiments

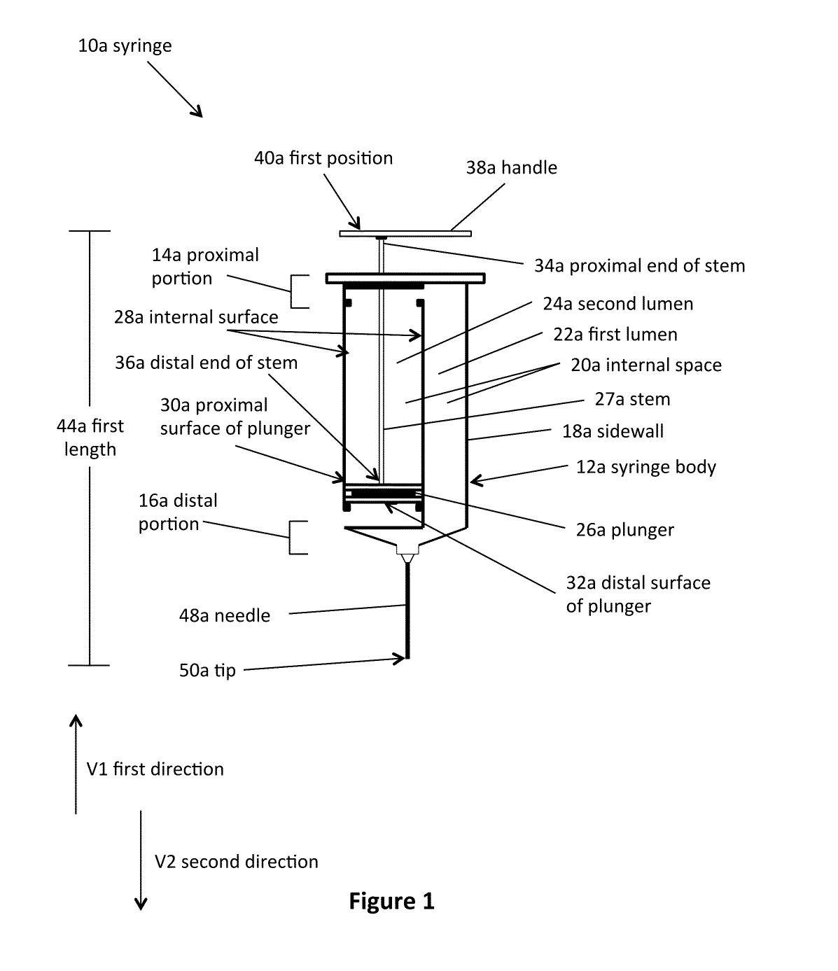

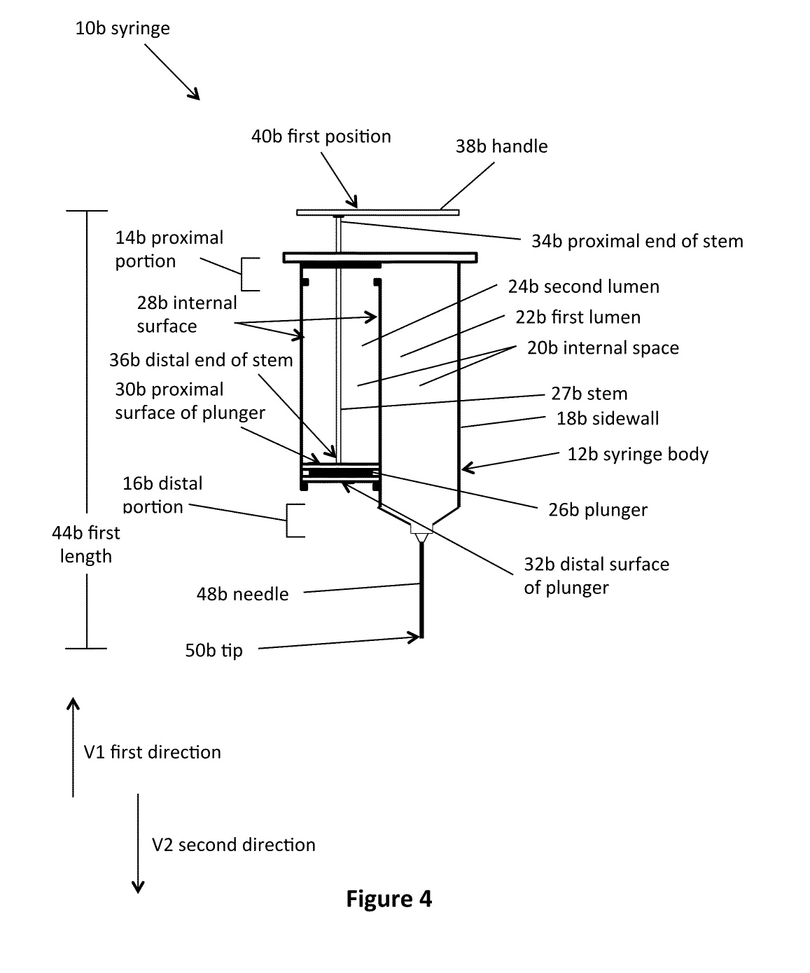

Syringes can be used to collect fluid, such as liquid and/or gas, from a variety of sources, including people and animals. As illustrated in FIGS. 1 and 4, a syringe 10a, 10b can include a syringe body 12a, 12b that may be elongate along a first direction V1 or a second direction V2. The syringe body 12a, 12b can define a proximal portion 14a, 14b and a distal portion 16a, 16b that is opposite the proximal portion 12a, 12b. It should be appreciated that the proximal portion 14a, 14b and the distal portion 16a, 16b can refer to portions of the syringe body 12a, 12b that may span a distance, and not just define a discrete point or end of the syringe body 12a, 12b, as shown in FIGS. 1 and 4. As well, the proximal portion 14a, 14b can be referred to as a top portion of the syringe body 12a, 12b, while the distal portion 16a, 16b can be referred to as a bottom portion of the syringe body 12a, 12b.

The syringe 10a, 10b can also include a sidewall 18a, 18b that extends from the proximal portion 14a, 14b to the distal portion 16a, 16b. In this manner, the sidewall can be configured to define a perimeter of the syringe 18a, 18b. The sidewall 18a, 18b can include various features, not illustrated, such as graduation marks for measuring a volume of liquid. As well, the sidewall 18a, 18b can also define other features that will be described further along.

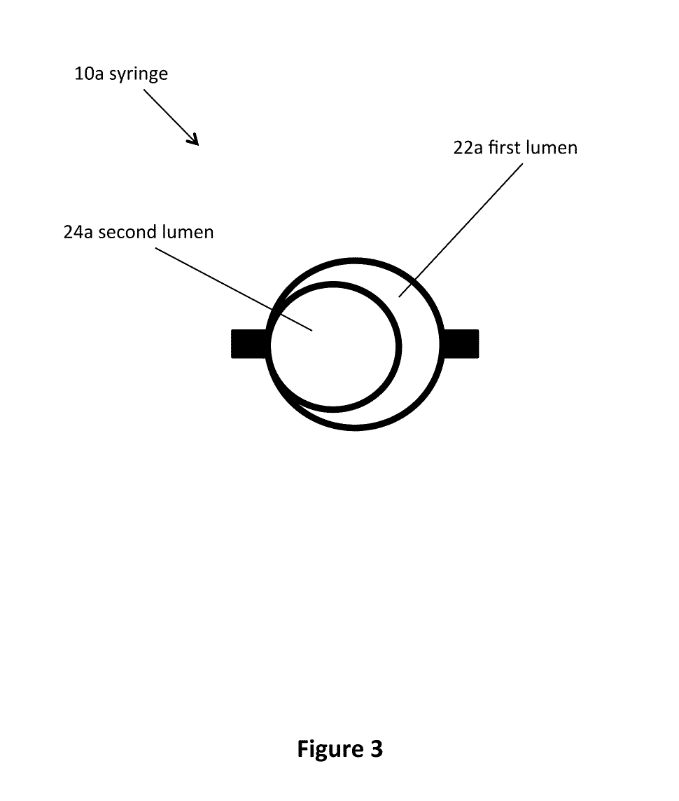

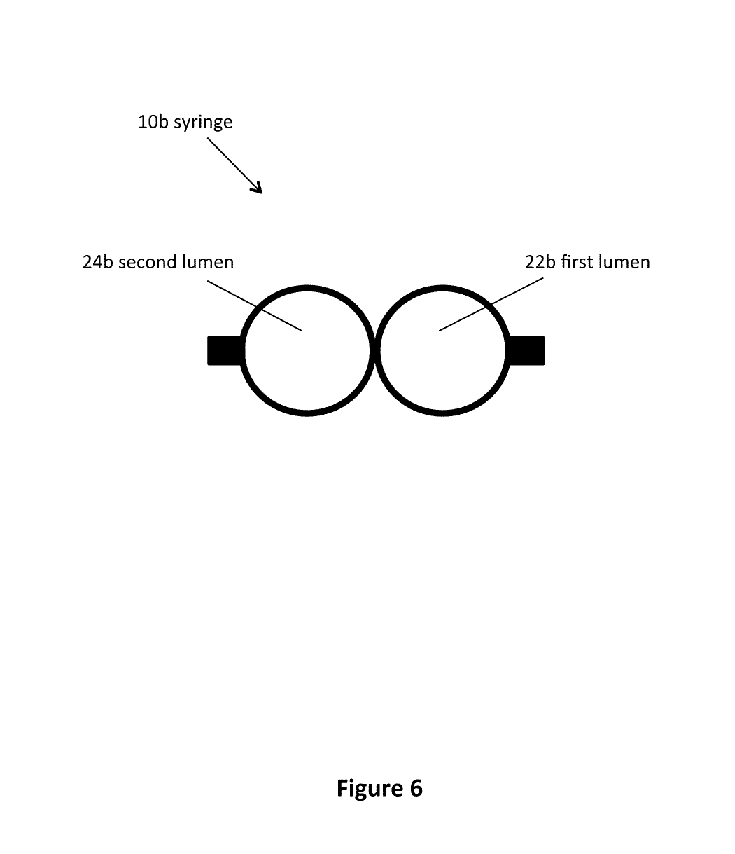

The syringe body 12a, 12b can also include an internal space 20a, 20b. Within the internal space 20a, 20b, the syringe 10a, 10b can further include a first lumen 22a, 22b and a second lumen 24a, 24b. It should be appreciated that the syringe 10a, 10b can be implemented with a variety of lumen configurations. For example, as shown in FIGS. 1-3, the second lumen 24a can be disposed within an internal space of the first lumen 22a. As well, in the embodiment shown in FIGS. 4-6, the second lumen 24b can be disposed side-by-side the first lumen 22b, such that the second lumen 24a, 24b is not located within the internal space of the first lumen 22a, 22b. Generally, the first lumen 22a, 22b and second lumen 24a, 24b can be laid out in any configuration with respect to each other and/or the syringe body 12a, 12b. Moreover, the first lumen 22a, 22b and second lumen 24a, 24b can be configured to define any size and/or shape. As well, it should be appreciated that some syringe embodiments can include three or more lumens.

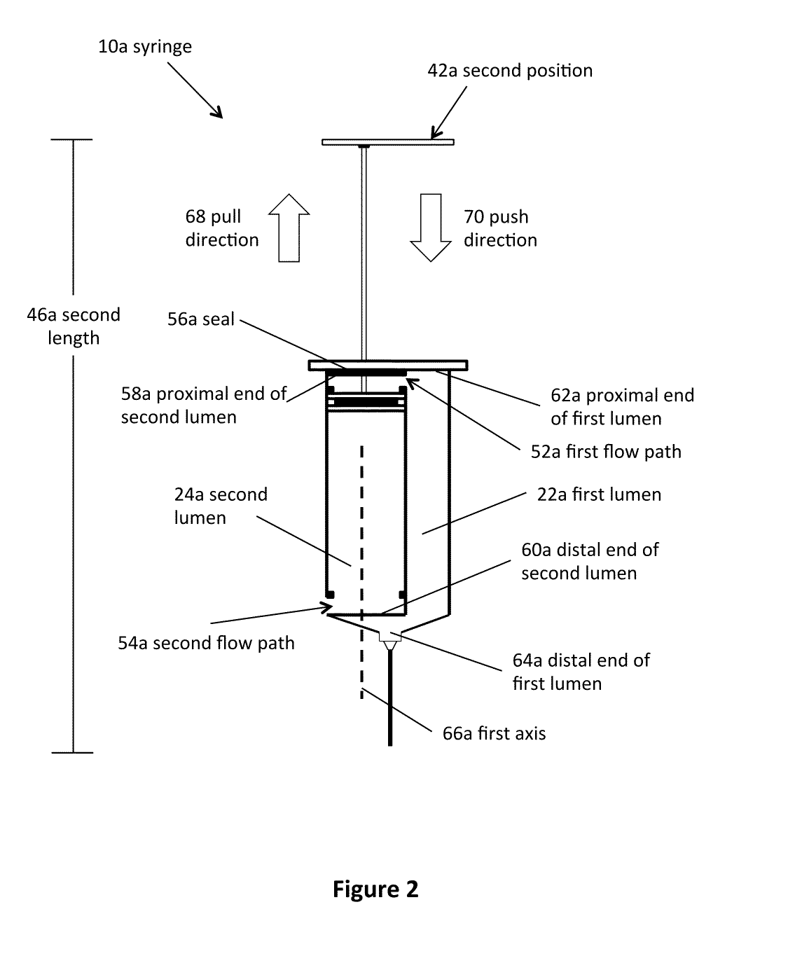

With specific reference to FIG. 2, the first lumen 22a can include a proximal portion 62a and a distal portion 64a that is opposite the proximal portion 62a. As well, the second lumen 24a can include a proximal portion 58a and a distal portion 60a that is opposite the proximal portion 58a. In some embodiments, the distal portion 64a of the first lumen 22a is distal to the distal portion 60a of the second lumen 24a. Furthermore, the syringe 10a can define a first axis 66a that is parallel to the first direction V1, whereby the first axis 66a can pass through at least a portion of the second lumen 24a and at least a portion of the first lumen 22a (as shown in FIG. 2).

As shown in FIGS. 1 and 4, the syringe 10a, 10b can also include a plunger 26a, 26b. The plunger 26a, 26b can be slideably received along an internal surface 28a, 28b of the second lumen 24a, 24b. As well, the plunger 26a, 26b can define a proximal surface 30a, 30b and a distal surface 32a, 32b that is opposite the proximal surface 30a, 30b. The plunger 26a, 26b can be configured to move along a first direction V1 and a second direction V2 that is opposite the first direction V1. In other words, the plunger 26a, 26b can move towards the distal portion 16a, 16b and away from the distal portion 16a, 16b. It should be appreciated that the plunger 26a, 26b can create a seal to substantially prevent fluid communication between the proximal surface 30a, 30b of the plunger 26a, 26b and the distal surface 32a, 32b of the plunger 26a, 26b.

The syringe 10a, 10b can also include a stem 27a, 27b that is elongate along the first direction V1. It can also be said that the stem 27a, 27b is elongate along the second direction V2. The stem 27a, 27b can define a proximal end 34a, 34b and a distal end 36a, 36b that is opposite the proximal end 34a, 34b. In this regard, the distal end 36a, 36b of the stem 27a, 27b can be coupled to the proximal surface 30a, 30b of the plunger 26a, 26b. Furthermore, the stem 27a, 27b can be configured to move along the first direction V1 and the second direction V2. Accordingly, it should be appreciated that because the stem 27a, 27b can be coupled to the plunger 26a, 26b, the stem 27a, 27b and the plunger 26a, 26b thereby move together.

The syringe 10a 10b can also include a handle 38a, 38b that can be coupled to the proximal end 34a, 34b of the stem 27a, 27b. As well, because the handle 38a, 38b, stem 27a, 27b and plunger 26a, 26b are effectively coupled together, the handle 38a, 38b can be configured to move along the first direction V1 and the second direction V2 together with the stem 27a, 27b and plunger 26a, 26b.

Accordingly, the handle 38a, 38b, stem 27a, 27b and plunger 26a, 26b can be configured to move from a first position 40a, 40b to a second position 42a, 42b. To move from the first position 40a, 40b to the second position 42a, 42b, the handle 38a, 38b can be pulled along a pull direction 68, which can be the same direction as the first direction V1. As well, to move from the second position 42a, 42b to the first position 40a, 40b, the handle can be pushed along the push direction 70, which can be the same direction as the second direction V2.

When the handle 38a, 38b and plunger 26a, 26b are in the first position 40a, 40b, the syringe 10a, 10b can define a first length 44a, 44b. Accordingly, when the handle 38a, 38b and plunger 26a, 26b are in the second position 42a, 42b, the syringe 10a, 10b can define a second length 46a, 46b. It should be appreciated that the second length 46a, 46b can be greater than the first length 44a, 44b.

As well, the syringe 10a, 10b can include a needle 48a, 48b that can be coupled to the distal portion 16a, 16b of the syringe body 12a, 12b. The needle 48a, 48b can be configured to receive fluid through a tip 50a, 50b of the needle 48a, 48b. When the tip 50a, 50b receives fluid, the fluid can move from the tip 50a, 50b into the needle 48a, 48b and into the first lumen 22a, 22b. As well, when the tip 50a, 50b expels fluid, the fluid can move from the first lumen 22a, 22b into the needle 48a, 48b and out the tip 50a, 50b.

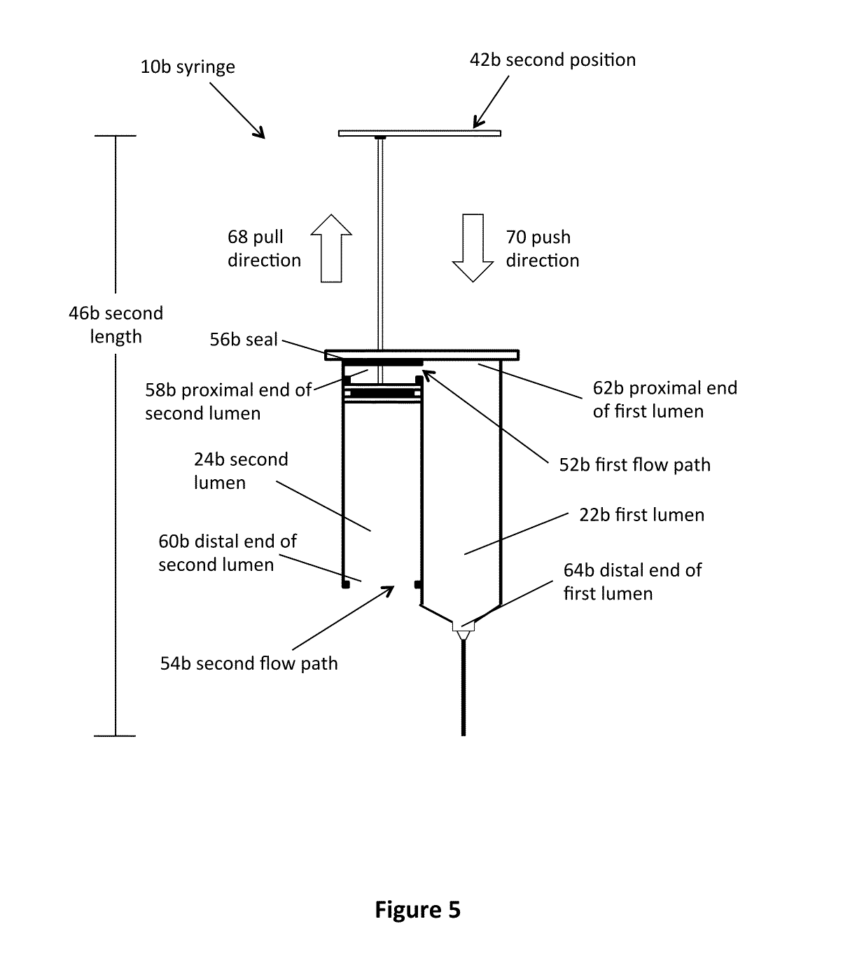

As shown in FIGS. 2 and 5, the syringe 10a, 10b can be configured to define various flow paths, which can allow fluid communication between specific portions of the syringe 10a, 10b and/or the outside environment. For example, as illustrated in FIGS. 2 and 5, the syringe body 12a, 12b can define a first flow path 52a, 52b that can allow fluid communication between the first lumen 22a, 22b and the second lumen 24a, 24b. Accordingly, when the plunger 26a, 26b and handle 38a, 38b are moved from the second position 42a, 42b to the first position 40a, 40b, air can move from the first lumen 22a, 22b through the first flow path 52a, 52b and into the second lumen 24a, 24b. In this regard, if the tip 50a, 50b is subcutaneous within a person or animal, the movement of the plunger 26a, 26b from the second position 42a, 42b to the first position 40a, 40b can thereby create a negative pressure, or vacuum, which can pull fluid from the needle 48a, 48b into the first lumen 24a, 24b.

Accordingly, when the plunger 26a, 26b and handle 38a, 38b are moved from the first position 40a, 40b to the second position 42a, 42b, this can create a positive pressure at the proximal surface 30a, 30b of the plunger. In this manner, as the handle 38a, 38b moves to the second position 42, 42b air can move from the second lumen 24a, 24b through the first flow path 52a, 52b and into the first lumen 22a, 22b. Accordingly, the movement of the air from the second lumen 24a, 24b to the first lumen 22a, 22b can push the liquid from the first lumen 22a, 22b to the needle 48a, 48b whereby the liquid can be expelled through the tip 50a, 50b. It should be appreciated that if the needle 48a, 48b is not attached to the syringe 10a, 10b, the fluid can thereby move through the first lumen 22a, 22b to an aperture at a distal portion 16a, 16b of the first lumen 22a, 22b.

It should be appreciated that the first flow path 52a, 52b can be disposed at any location within the syringe 10a, 10b. For example, in some embodiments, the first flow path 52a, 52b is disposed at a proximal portion 14a, 14b of the syringe body 12a, 12b.

As well, the syringe 10a, 10b can include a second flow path 54a, 54b that provides air flow path between the second lumen 24a, 24b and the outside environment. Accordingly, when the plunger 26a, 26b, stem 27a, 27b and handle 38a, 38b move from the second position 42a, 42b to the first position 40a, 40b, air can move from the second lumen 24a, 24b through the second flow path 54a, 54b to thereby create a negative pressure, or vacuum, to pull fluid from the needle 48a, 48b into the first lumen 22a, 22b.

As such, when the plunger 26a, 26b, stem 27a, 27b and handle 38a, 38b move from the first position 40a, 40b to the second position 42a, 42b, air can move from through the second flow path 42a, 42b into the second lumen 24a, 24b, to thereby create a positive pressure within the first lumen 22a, 22b to thereby push the fluid from the first lumen 22a, 22b to the needle 48a, 48b and into the outside environment. Furthermore, as shown in FIGS. 2 and 5, the second flow path 54a, 54b can be an aperture disposed at a distal portion 16a, 16b of the syringe body 12a, 12b. However, this is just one example and it should be appreciated that the second flow path 54a, 54b can be disposed at any location on the syringe body 12a, 12b.

With continued reference to FIGS. 2 and 5, the syringe 10a, 10b can also include a seal 56a, 56b disposed within an internal space of the second lumen 24a, 24b. Specifically, the seal 56a, 56b can be disposed along a proximal portion 14a, 14b of the second lumen 24a, 24b. The seal 56a, 56b can be configured to slideably and sealably receive the stem 27a, 27b. For example, the seal 56a, 56b can include an aperture that can allow the stem 27a, 27b to slideably move within. In this manner, the seal can be configured to provide a vacuum-tight sealing engagement with the stem 27a, 27b such that fluid and/or air does not substantially leak from the proximal portion 14a, 14b of the syringe 10a, 10b and into the environment. It should be appreciated that within the context of this disclosure the term substantially means “for the most part.”

### Method Embodiments

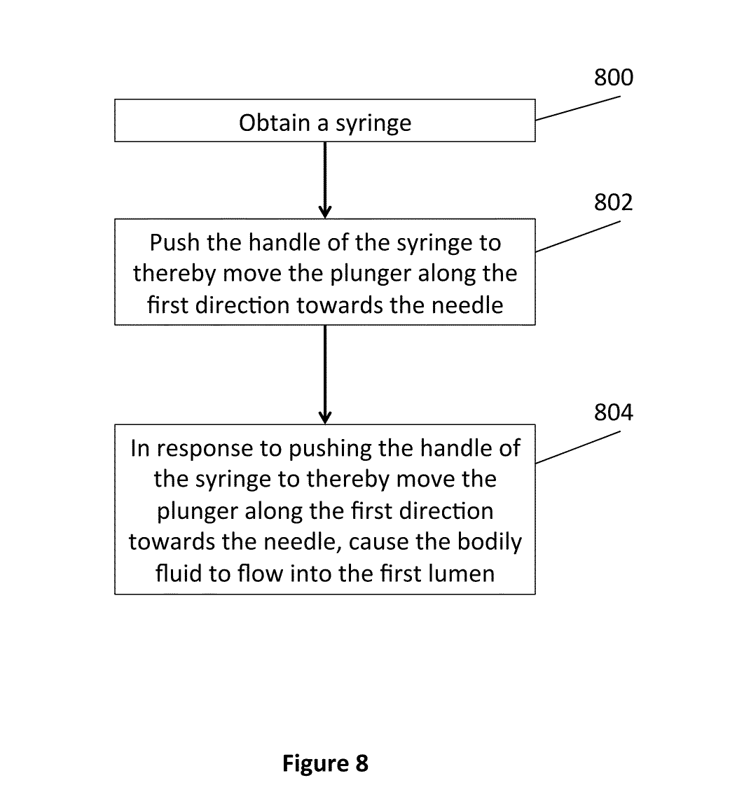

This disclosure also includes various methods of using the syringe 10a, 10b as described. For example, the disclosure includes methods for removing fluid from a patient. As shown in FIG. 8, the method can include obtaining the syringe 10a, 10b (at step 800). The method can also include pushing the handle 38a, 38b of the syringe 10a, 10b to thereby move the plunger 26a, 26b along the first direction V1 towards the needle 48a, 48b (at step 802). In response to pushing the handle 38a, 38b of the syringe 10a, 10b to thereby move the plunger 26a, 26b along the first direction V1 towards the needle 48a, 48b, the method can include causing the fluid to flow into the first lumen 22a, 22b (at step 804). The fluid can thereby flow from the tip 50a, 50b of the needle 48a, 48b into the first lumen 22a, 22b along the second direction V2. In this regard, the method may include pushing the handle 38a, 38b to create a vacuum to thereby draw fluid into the first lumen 22a, 22b.

The method may also include causing the first lumen 22a, 22b to fill with fluid. In this regard, the fluid can enter the first lumen 22a, 22b from the distal portion of the first lumen 22a, 22b, whereby the distal portion is adjacent the needle 48a, 48b. As the handle 38a, 38b is pushed, the fluid can rise from the distal portion of the first lumen 22a, 22b towards the proximal portion of the first lumen, whereby the proximal portion of the first lumen is adjacent the seal 56a, 56b and the handle 38a, 38b.

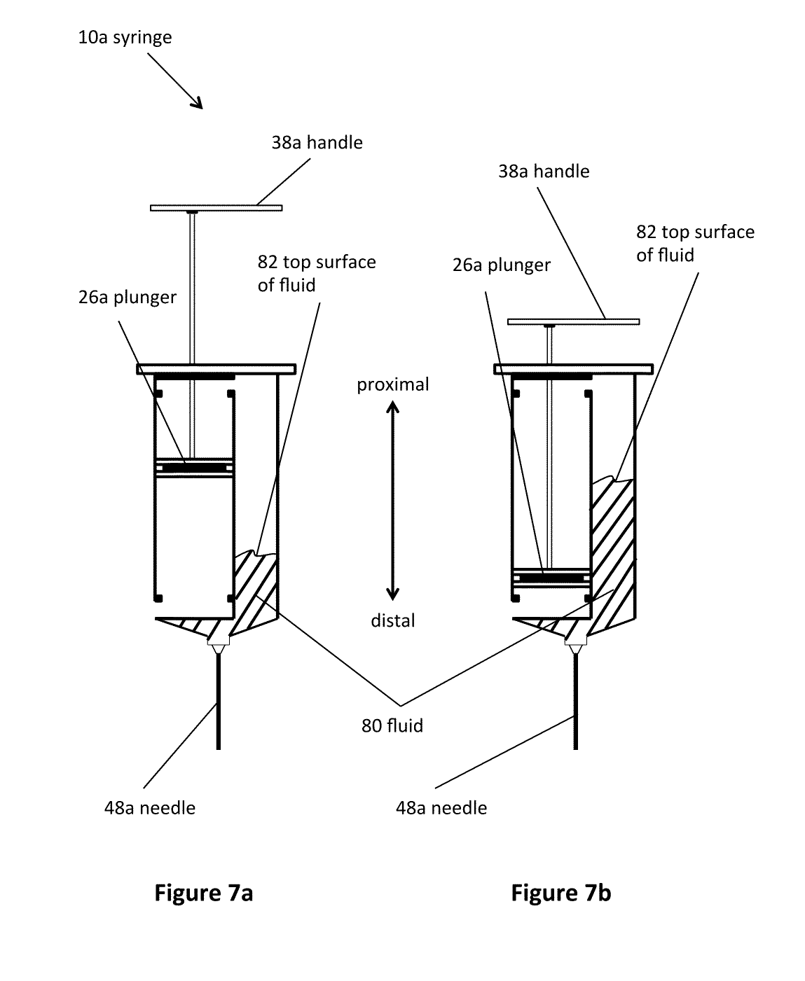

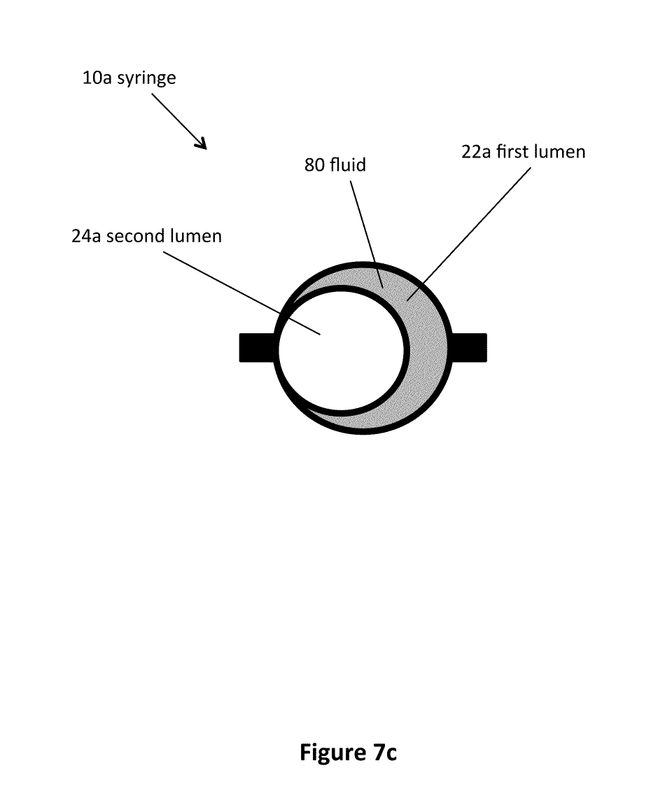

As shown in FIG. 7a, as the fluid 80 enters the needle 48a, a top surface 82 of the fluid 80 can be located distal to the plunger 26a. The method can further include pushing the handle 38a of the syringe 10a along the first direction V1 until the plunger 26a is located distal to the top surface 82 of the fluid 80 (as shown in FIG. 7b). Moreover, the distal direction is the same as the second direction V2, and the proximal direction is the same as the first direction V1.

The manner in which the fluid is received within the first lumen can be described with regard to specific syringe configurations, such as syringe 10a. For example, as illustrated in FIG. 7c, the method may include causing at least a portion of the fluid 80 to surround at least a portion of the second lumen 24a. FIG. 7c shows the fluid surround a majority (i.e. greater than 50%) of the perimeter of the second lumen 24a.

The method may also include steps to expel fluid 80 from the syringe 10a, 10b. For example, the method may include pulling the handle 38a, 38b of the syringe 10a, 10b to thereby move the plunger 26a, 26b along the second direction V2. In response to pulling the handle 38a, 38b of the syringe 10a, 10b to thereby move the plunger 26a, 26b along the second direction V2, the method may also include causing the fluid 80 to flow from the first lumen 22a, 22b to the tip 50a, 50b of the needle 48a, 48b along the first direction V1.

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. A syringe comprising:
a syringe body elongate along a first direction, wherein the syringe body defines a proximal portion, a distal portion that is opposite the proximal portion, a sidewall that extends between the proximal and distal portions, and an internal space;
a first lumen disposed within the internal space of the syringe body;
a second lumen disposed within the internal space of the syringe body;
a plunger slideably received along an internal surface of the second lumen, wherein the plunger defines a proximal surface and a distal surface that is opposite the proximal surface, and wherein the plunger is configured to move along a first direction and a second direction that is opposite the first direction;
a stem elongate along the first direction, wherein the stem has a proximal end and a distal end that is opposite the proximal end, and wherein the distal end of the stem is coupled to the proximal end of the plunger, wherein the stem is configured to move along the first direction and the second direction;
a handle coupled to the proximal end of the stem, wherein the handle is configured to move along the first direction and the second direction, wherein the handle and plunger are configured to move from a first position to a second position, wherein when the handle and plunger are in the first position, the syringe defines a first length, wherein when the handle and plunger are in the second position, the syringe defines a second length, and wherein the second length is greater than the first length; and
a needle coupled to the distal portion of the syringe body, wherein the needle is configured to receive fluid through a tip of the needle, and wherein the fluid moves from the needle into the first lumen.

2. The syringe of claim 1, wherein the second lumen is disposed within an internal space of the first lumen.

3. The syringe of claim 2, wherein the syringe body defines a first flow path between the first lumen and the second lumen, and wherein when the plunger and handle move from the second position to the first position, air moves from the first lumen through the first flow path and to the second lumen to thereby pull the fluid from the needle into the first lumen.

4. The syringe of claim 3, wherein the first flow path is disposed at a proximal portion of the syringe body.

5. The syringe of claim 4, wherein the sidewall of the syringe body defines a second flow path, wherein the second flow path provides air flow path between the second lumen and the outside air, and wherein when the plunger and handle move from the second position to the first position, air moves from the second lumen through the second flow path to thereby pull the fluid from the needle into the first lumen.

6. The syringe of claim 5, wherein the second flow path is an aperture disposed at a distal portion of the syringe body.

7. The syringe of claim 6, further comprising a seal disposed within an internal space of the second lumen along a proximal portion of the second lumen, wherein the seal slideably and sealably receives the stem, and wherein the seal is configured to provide a vacuum-tight sealing engagement.

8. The syringe of claim 2, wherein the first lumen comprises a proximal portion and a distal portion that is opposite the proximal portion, wherein the second lumen comprises a proximal portion and a distal portion that is opposite the proximal portion, and wherein the distal portion of the first lumen is distal to the distal portion of the second lumen.

9. The syringe body of claim 8, wherein a first axis that is parallel to the first direction passes through at least a portion of the second lumen and at least a portion of the first lumen.

10. A method for removing fluid from a patient, comprising:
obtaining a syringe having a syringe body; a first lumen disposed within an internal space of the syringe body; a second lumen disposed within the internal space of the syringe body; a plunger slideably received along an internal surface of the second lumen; a stem elongate along a first direction, wherein the stem is configured to move along the first direction and a second direction that is opposite the first direction; a handle coupled to the stem, wherein the handle is configured to move along the first direction and the second direction; and a needle coupled to the syringe body, wherein the needle is configured to receive the fluid through a tip of the needle;
pushing the handle of the syringe to thereby move the plunger along the first direction towards the needle; and
in response to pushing the handle of the syringe to thereby move the plunger along the first direction towards the needle, causing the fluid to flow into the first lumen, wherein the fluid flows from the tip of the needle into the first lumen along the second direction.

11. The method of claim 10, further comprising causing the first lumen to fill with fluid, wherein the fluid enters the first lumen from the distal portion of the first lumen, and wherein as the handle is pushed, the fluid rises from the distal portion of the first lumen towards the proximal portion of the first lumen.

12. The method of claim 11, wherein as the fluid enters the needle, a top surface of the fluid is located distal to the plunger, the method further comprising pushing the handle of the syringe along the first direction until the plunger is located distal to the top surface of the fluid.

13. The method of claim 12, further comprising causing at least a portion of the fluid to surround at least a portion of the second lumen.

14. The method of claim 13, further comprising:
pulling the handle of the syringe to thereby move the plunger along the second direction; and
in response to pulling the handle of the syringe to thereby move the plunger along the second direction, causing the fluid to flow from the first lumen to the tip of the needle along the first direction.

15. The method of claim 14, wherein the syringe includes a seal disposed within an internal space of the second lumen along a proximal portion of the second lumen, wherein the seal slideably and sealably receives the stem such that the seal provides a vacuum-tight sealing engagement with the stem, the method further comprising pushing the handle of the syringe to create a vacuum to thereby draw fluid into the first lumen.
